(19) United States
(12) Patent Application Publication
(10) Pub. No.: US 2003/0099949 A1
(43) Pub. Date: May 29, 2003

(54) Arrays having clustered arrangements and methods of making and using

(75) Inventors: Patrick E. Guire, Eden Prairie, MN (US); Kristin S. Taton, Little Canada, MN (US)

Correspondence Address: MERCHANT & GOULD PC, P.O. BOX 2903, MINNEAPOLIS, MN, 55402-0903, US

(73) Assignee: Surmodics, Inc.

(21) Appl. No.: 09/972,116

(22) Filed: Oct. 5, 2001

(51) Int. Cl. C12Q 1/68; G01N 33/53; G01N 33/542; B05D 3/00

(52) U.S. Cl. 435/6; 435/7.9; 427/2.11

(57) ABSTRACT

Arrays including microparticles having probe moieties are used for the detection of a target in a sample. Microparticles are immobilized in clustered arrangements on at least a portion of a substrate. A detection scheme is performed to detect a marker associated with the target which can be bound to a probe of a clustered arrangement.

### FIELD OF THE INVENTION

This invention relates to the field of arrays for use in detecting targets suspected to be present in a sample. More particularly, the invention relates to arrays having clustered arrangements of microparticles immobilized on a substrate.

### BACKGROUND OF THE INVENTION

In the past several years, a new technology, called the DNA array, has attracted interest among biologists. This technology promises to monitor part or all of an organism's genome on a single chip so that researchers can develop a better picture of the interactions among hundreds or thousands of genes simultaneously. This technology has been termed biochip, DNA chip, DNA microarray, gene array, and genome chip. Generally, a DNA array relies upon standard base pairing rules developed by Watson and Crick to analyze the presence, or the sequence, of a particular complementary nucleic acid sequence.

More recently, attention has focused on fabrication of protein or peptide arrays, and this area is commonly referred to as “proteomics.” In one example of this approach, a library of peptides can be used as probes to screen for drugs. The peptides can be exposed to a receptor, and those probes that bind to the receptor can be identified. In one application, more than 10,000 protein spots were printed on a glass slide. The chip was used to identify protein-protein and protein-drug interactions. (G. MacBeath and S. L. Schreiber, 2000, Printing Proteins as Microarrays for High-Throughput Function Determination, Science 189:1760-1763).

In more recent years, the demand for high-throughput and cost-effective analysis of complex mixtures has driven technology toward the fabrication of compact, high-density array devices. These arrays are fabricated using conventional techniques such as ink-jet printing, screen printing, photolithography, and photodeposition, in which the sensing chemistries are applied directly to the sensor surface. Typically, an array is fabricated by attaching a nucleic acid or peptide directly to a substrate. Typically, multiple fabrication steps are required that are labor intensive and subject to some degree of variability.

Given current fabrication schemes, the precise location of a probe on the surface of an array must be known prior to interrogating a sample. Therefore, fabrication of the arrays relies upon such techniques as printing or spotting of the probe onto the surface of the array, so that the addresses or locations of each probe is known prior to use of the array. Once the complexes are detected, the location of the complex is compared to the mapped surface of the array, and the identity of the target is determined.

### SUMMARY OF THE INVENTION

Generally, the invention is directed towards arrays fabricated by disposing microparticles, which are coupled to probes, on a substrate. The arrays can be used to determine the presence of a target in a sample. Arrays are formed by preparing a slurry which includes at least one plurality of microparticles and a matrix-forming material and disposing the slurry on a substrate. In one embodiment, the matrix-forming material of the slurry is a photoreactive polymer. In each plurality of microparticles, the microparticles are coupled to a particular probe. In one embodiment, the probe is a nucleic acid molecule. In other embodiments, the probe is a protein molecule, for example an antibody, a fragment of an antibody, or a member of a binding pair. The slurry is disposed on a substrate, and then treated to form a matrix in which the microparticles become immobilized, thereby forming a clustered arrangement of microparticles at a desired location on the substrate. In one embodiment, the microparticles become entrapped by the matrix, wherein the entrapment of the microparticles is primarily dependent on the physical constraints of the matrix on the microparticles and not dependent on covalent or ionic bonding interactions between the microparticles and the matrix. Typically, an array is created by disposing slurries at different locations on the substrate, each slurry containing a plurality of microparticles attached to a unique probe. In an assay to determine the presence of a target in a sample, the target is typically labeled with a detectable marker and the sample and array are maintained under conditions to allow specific binding of the detectable marker-labeled target to the probe, which is coupled to the microparticles of a particular cluster. A detection step is performed to determine the presence of the detectable marker-labeled target associated with the clustered arrangement of microparticles.

### BRIEF DESCRIPTION OF THE DRAWINGS

FIG. 1 is a schematic diagram of a coating of microparticles in a matrix immobilized on a substrate.

FIGS. 2a-2d are photomicrographs of microparticles immobilized within a matrix on a substrate.

FIGS. 3a and 3b are photomicrographs of microparticles immobilized within a matrix on a substrate.

FIGS. 4a-4c are photomicrographs of microparticles immobilized within a matrix on a substrate. FIG. 4c shows hybridization of a labeled target on the microparticles.

FIGS. 5a-5e are photomicrographs of microparticles immobilized within a matrix on a substrate.

### DETAILED DESCRIPTION OF THE PREFERRED EMBODIMENTS

As used herein, a “probe” is a moiety that is to be immobilized on a substrate to form an array. Typically, according to the invention, the probe is coupled to a microparticle, and the microparticle, in turn, is immobilized on a substrate, thereby forming an array. The probe can be a molecule, a particle, or a cell that can specifically interact with a particular target.

As used herein, a “target” refers to a molecule, a cell, or a particle suspected to be present in a sample and that can specifically interact with a particular probe.

The term “sample” is used in its broadest sense. The term includes a specimen or culture suspected of containing target.

As used herein, the term “matrix” refers to a three-dimensional network of linked network of molecules, (herein referred to as “matrix-forming material”) that can surround and encompass the microparticles of the invention.

As used herein, “coupling” refers to the direct or indirect attachment of one moiety to another moiety through the formation of at least one chemical bond, which include covalent, ionic, coordinative, hydrogen, or Van der Waals bonds, or non-chemical interactions. For example, coupling of compound “A” to compound “D” can be direct and involve the formation of a covalent bond between “A” and “D”, or coupling of compound “A” to compound “D” can be indirect and involve the presence of compound “B” and “C” where coordinative bonds exist between “A” and “B”, and “C” and “D”, and a covalent bond exists between “B” and “C”. It is understood that according to this description that two moieties can be coupled together in a variety of ways. Such coupling can include, but is not limited to, specific non-covalent streptavidin- or avidin- to biotin interactions and hapten-to-antibody interactions; hydrophobic interaction; magnetic interaction; polar interactions, such as “wetting” associations between two polar surfaces or between oligonucleotide/polyethylene glycol; formation of a covalent bond, such as an amide bond, disulfide bond, thioether bond, ether bond, carbon-carbon bond, or via other crosslinking agents; and via an acid-labile linker. As used herein, “bonding” refers to the direct attachment of two moieties typically through covalent or ionic bonding.

As used herein the term “cluster” or “clustered arrangement” or “clustered arrangement” of microparticles” refers to a plurality of similar microparticles that are immobilized in a group on the surface of a substrate. The clustered arrangements of microparticles are formed by the entrapment of the microparticles in a polymeric matrix which is disposed on a substrate. An array typically comprises a plurality of clustered arrangements of microparticles, the clusters being spatially separated from each other on the array. The microparticles of a distinct clustered arrangement typically are coupled to the same probe moiety.

As used herein, “immobilization” refers to the positional fixation of microparticles of a clustered arrangement on a substrate.

In a one embodiment, the microparticles are immobilized on the substrate by entrapment in a polymeric matrix. As used herein, “entrapment”, “entrapped”, or “entrapping” refers to the positional fixation of microparticles within the polymer matrix on the substrate where the positional fixation is primarily due to the physical constraint of the microparticles by the network of polymeric strands and is not dependent on covalent or ionic chemical bonding interactions between the microparticle and the substrate or between the microparticles and the polymer. Although in some cases it may be possible for some covalent or ionic bonds to be formed between the microparticles or agent-coupled microparticles and the polymeric material, entrapment is primarily dependent on physical factors established by the material of the matrix which constrains the microparticles, holding them in the matrix.

As used herein, “slurry” refers to a mixture including a matrix-forming material and microparticles.

The present invention provides a method for detecting target in a sample using clustered arrangements of microparticles. At least one clustered arrangement of microparticles is present on a substrate thereby forming an array and the microparticles within a particular cluster are coupled to a unique probe. The microparticles of a clustered arrangement are typically immobilized in a matrix on a substrate. Clustered arrangements of microparticles on a substrate can be formed by mixing microparticles that are coupled to a particular probe with a matrix-forming material, for example, a crosslinkable polymer, disposing the microparticles onto a substrate by spotting or a similar procedure, and treating the material to form a matrix, thereby immobilizing the microparticles on the substrate. A target, if present in a sample, can be coupled to a target marker and can specifically bind to probe coupled to microparticles that are in a clustered arrangement. The array can provide a plurality of clustered arrangements of microparticles, the microparticles of each clustered arrangement being typically coupled to a particular probe, therefore, the array can comprise a plurality of different probes. In some embodiments, the microparticles of a clustered arrangement can also be coupled to a microparticle marker.

Sample suspected to contain target can be treated to couple target marker to the target. The sample can then be applied to the array, so that target marker-labeled target, if present in the sample, will specifically interact with probe coupled to the microparticles of a particular clustered arrangement provided by the array. Thereafter, a detection scheme is performed to detect bound target marker-labeled target; optionally, if a microparticle marker is present and coupled to the microparticles of a clustered arrangement, this can also be detected. Determination of the location of the clustered arrangement of microparticles or detection of the microparticle marker allows for determination of the probe.

The invention contemplates methods for preparation of arrays containing clustered arrangements of microparticles, and the arrays themselves. The invention also contemplates detecting target using the arrays comprising clustered arrangements of microparticles. Also contemplated are kits for detecting target in a sample.

Arrays prepared according to the invention are fabricated on a solid support, also referred to herein as a substrate. Generally, the term “solid support” or “substrate” refers to a material that provides at least a two-dimensional surface on which the microparticles of the invention can be immobilized. The composition of the solid support can be any sort of suitable material to which the clustered arrangement of microparticles can be directly or indirectly immobilized. The composition of the substrate can vary, depending upon the composition of materials used to prepare the cluster, which typically includes microparticles and polymeric material.

Preferably, the substrate surface does not interfere with target binding and is not subject to high amounts of non-specific binding. Suitable materials for the substrate include biological or nonbiological, organic or inorganic materials. Suitable solid substrates include, but are not limited to, those made of plastics, functionalized ceramic, resins, polysaccharides, functionalized silica, or silica-based materials, functionalized glass, functionalized metals, films, gels, membranes, nylon, natural fibers such as silk, wool and cotton and polymers.

Preferably, the substrate comprises an integral surface for immobilization of the clustered arrangements of microparticles. The substrate can be either “substantially flat”, meaning that the surface is substantially planar and has little or no surface configurations, or the substrate can have surface configurations such as raised portions, surface projections, etched areas, wells, and the like. A raised area can be useful during steps of detecting a target in a sample. The surface of the substrate preferably is provided by a single substrate.

Optionally, portions of the substrate can be pre-treated with compounds to develop areas having different surface properties, for example, different regions of hydrophobicity or hydrophilicity. “Hydrophilic” and “hydrophobic” are used herein to describe compositions broadly as water attracting and water repelling, respectively. Generally, hydrophilic compounds are relatively polar and often ionizable. Such compounds usually bind water molecules strongly. Hydrophobic compounds are usually relatively non-polar and non-ionizing. “Hydrophobic” refers to materials or surfaces that have a low affinity for water, are not readily mixed with or wetted by water, and which are generally water-repellant. Hydrophobic and hydrophilic are relative terms and are used herein in the sense that various compositions, liquids and surfaces can be hydrophobic or hydrophilic relative to one another.

The dimensions of the substrate can vary and can be determined by such factors as the dimensions of the desired array, and the amount of probe diversity desired. In another embodiment, multiple arrays can be provided on a single substrate, and the dimensions of a substrate in this instance can be influenced by such factors as the diversity of arrays provided on the substrate, and the size of the arrays on the substrate.

In one embodiment, the substrate can be pre-coated with an organosilane material. Pre-coating with an organosilane material can be useful in providing a surface of the substrate that can form bonds with matrix-forming material, if desired. In this embodiment, the substrate is cleaned, pretreated or cleaned and pretreated prior to attachment of the microparticles. The substrate (for example, a soda lime glass microscope slide) is silane treated by dipping it in a mixture of 1% p-tolydimethylchlorosilane (T-silane) and 1% N-decyldimethylchlorosilane (D-silane, United Chemical Technologies, Bristol, Pa.) in acetone, for 1 minute. After air drying, the slides are cured in an oven at 120° C. for one hour. The slides are then washed with acetone followed by dipping in distilled water. The slides are further dried in an oven for 5-10 minutes. Other pretreatment or washing steps will be apparent upon review of this disclosure. Optionally, portions of the substrate can be pre-treated with compounds to develop areas having different surface properties, for example, different regions of hydrophobicity or hydrophilicity.

In another embodiment, the substrate can be functionalized with a reactive compound. A slurry, which includes a matrix-forming material and microparticles, can then be disposed on the substrate presenting the reactive compound. The substrate and slurry can be treated to couple the matrix-forming material to the substrate via the reactive groups and also to immobilize the microparticles by forming a matrix from the matrix-forming material.

The microparticles of the clustered arrangements can comprise any three-dimensional structure that can be resuspended in a slurry of matrix-forming material which can be disposed on a substrate. A clustered arrangement can include microparticles of any size or shape, or a mixture of different sizes and shapes of microparticles. Typically the microparticles are spherular in shape. As used herein “spherular” refers to three dimensional shapes that include, spherical, spheroidal, rounded, globular shapes and the like. Preferably, the microparticle is of a size in the range of about 100 nm to about 100 μm in diameter, more preferably in the range of 1 to 5 μm.

According to the invention, the microparticle can be fabricated from any suitable material. Suitable materials include, for example, polymers such as poly(methylmethacrylate), polystyrene, polyethylene, polypropylene, polyamide, polyester, polvinylidenedifluoride (PVDF), and the like; glass, including controlled pore glass (CPG) and silica (nonporous glass); metals such as gold, steel, silver, aluminum, silicon, copper, ferric oxide crystals, and the like; magnetite, and the like. Examples of useful microparticles are described in, for example, “Microparticle Detection Guide”, from Bangs Laboratories, Fishers, Ind.

In some embodiments it is preferable that the microparticles are swellable and can incorporate detectable compounds, for example, a fluorescent dye. As used herein, “swellable” refers to the ability of the microparticles to expand and become more porous when in an appropriate medium and can incorporate compounds or particles in a swollen state. Such swellable microparticles are typically composed of polystyrene or copolymers of polystyrene and are typically swellable in an organic solvent. In some embodiments, microparticles can be swollen and a fluorescent organic dye can be incorporated into the microparticle. In other embodiments the microparticles can be impregnated with a different detectable material, for example a magnetic material, or a combination of detectable materials.

Microparticles can also be obtained commercially, from, for example, Bangs Laboratories (Fishers, Ind.), Polysciences (Germany), Molecular Probes (Eugene, Oreg.), Duke Scientific Corporation (Palo Alto, Calif.), Seradyn Particle Technology (Indianapolis, Ind.), and Dynal Biotech (Oslo, Norway).

The microparticles of the invention can possess one or more desirable properties, such as ease of handling, dimensional stability, optical properties, a sufficient size to adequately couple the desired amount of probe to a substrate, and the like. The microparticles can be chosen to provide additional desired attributes, such as a satisfactory density, for example, a density greater then water or an other solvent used in fabrication of the array, or properties that allow the microparticle to be coupled to a probe or a detectable marker.

The microparticles can be modified to provide reactive groups for coupling one or more probes to the microparticle. In other embodiments, reactive groups can be provided on the microparticle to allow for coupling of a detectable marker to the microparticle, for coupling of the microparticles to each other, for coupling the microparticles to a matrix that is disposed on the substrate, for coupling the microparticles to the substrate, or any combination of the above. Suitable reactive groups can be chosen according to the nature of the moiety that is to be attached to the microparticle. Examples of suitable reactive groups include, but are not limited to, carboxylic acids, sulfonic acids, phosphoric acids, phosphonic acids, aldehyde groups, amine groups, thiol groups, thiol-reactive groups, epoxide groups, and the like. For example, carboxylate-functionalized microparticles can be used for covalent coupling of proteins and other amine-containing molecules using water-soluble carbodiimide reagents. Aldehyde-functionalized microparticles can be used to couple the microparticles to proteins and other amines under mild conditions. Amine-functionalized microparticles can be used to couple the microparticle to a variety of amine-reactive moieties, such as succinimidyl esters and isothiocyanates of haptens and drugs, or carboxylic acids of proteins. In some applications, sulfate-functionalized microparticles can be used to passively absorb a protein such as bovine serum albumin (BSA), IgG, avidin, streptavidin, and the like, onto the microparticles. In another embodiment, the reactive groups can be present on such binding partners as biotin, avidin, streptavidin, protein A. These and other modified microparticles are commercially available from a number of commercial sources, including Molecular Probes, Inc. (Eugene, Oreg.).

Another method for coupling moieties of the invention is through a combination of chemical and affinity interactions, herein referred to as “chemi-affinity” interactions, as described by Chumura et al. (2001, Proc. Natl. Acad. Sci., 98:8480). Binding pairs can be engineered that have high binding specificity and a neglible dissociation constant by functionalizing each member of the binding pair, near the affinity binding sites of the pair, with groups that will react to form a covalent bond. For example, the substituents of each functionalized member can react, for example by Michael addition or nucleophilic substitution, to form a covalent bond, for example a thioether bond. Antigen:anti-antigen antibody pairs, complementary nucleic acids, and carbohydrate:lectin pairs are example of binding pairs that can be functionalized to provide chemi-affinity binding pairs.

Microparticles can also be coupled to probes, detectable markers, or other species via crosslinking reagents. Commercially available crosslinking agents can be obtained from, for example, Pierce Chemical Company (Rockford, Ill.). Useful crosslinking agents include homobifunctional and heterobifunctional crosslinkers. Two nonlimiting examples of crosslinking agents that can be used on microparticles coated with, for example, proteins, are di-succinimidyl suberate and 1,4-bis-maleimidobutane.

In some embodiments the microparticle can also be coupled to a compound or moiety that enables detection of a bound target or increases the sensitivity of the detection step. For example, microparticles of a clustered arrangement can be coupled to both fluorescein and a probe. The probe on these microparticles can specifically bind an Alexa Fluor™ 488-labeled (Molecular Probes, Eugene, Oreg.) target. Upon binding of the Alexa Fluor™ 488-labeled target to the probe, the presence of both the fluorescein and Alexa Fluor™ 488 molecules on the same microparticle can synergistically increase the fluorescence emission from the microparticle in the clusters. In another example, tyramide signal amplification can be performed in a clustered arrangement of microparticles by coupling tyrosine residues to the microparticle, labeling or coupling the target to a horseradish peroxidase enzyme (HRP), binding the HPR-coupled target to the probe, and depositing a fluorescent tyramide derivative on the substrate, thereby causing the deposition of an activated tyramide derivative on the microparticles in a cluster. These methods can greatly increase the sensitivity of the array when an assay is performed.

According to the invention, an array comprises a substrate, at least one cluster of microparticles immobilized in a matrix on the substrate, and a probe coupled to microparticles of a clustered arrangement. As described herein, the probe comprises a moiety which can be recognized by a particular target, such as, for example, nucleic acid or peptides. The probe can be a molecule, a particle, or a cell that can specifically interact with a particular target. The probe can include naturally occurring or man-made molecules, and it can be used in its unaltered state or as aggregates with other species. Typically, the specific interaction of the probe and the target is based on chemical bonds that establish affinity interactions between the probe and target, for example, between an antibody and an antigen, or specific interactions based on an arrangement of repetitive hydrogen bonding patterns, for example between an oligonucleotide and its complementary oligonucleotide. In one embodiment, the probe comprises a biological molecule, such as, a nucleic acid. However, the probe can be any other molecule that specifically binds to a target. The probe can be a protein, such as an immunoglobulin, a cell receptor, such as a lectin, or a fragment thereof (e.g., F_{ab} fragment, F_{ab′} fragments, and the like). In another embodiment, the probe can include cells or particles, such as viral particles and the target can be a molecule, cell, or an other particle that interacts with the cells or particles. In this embodiment the cell or particle probe can display multiple specific interactions with targets. Given the teachings herein, one of skill in the art can select a desired probe, or set of desired probes, for fabrication of an array.

In one embodiment, the probe is a nucleic acid and the array includes a plurality of clustered arrangements of microparticles, the microparticles of each individual arrangement coupled to a unique nucleic acid. As used herein, the term “nucleic acid” refers to any of the group of polynucleotide compounds having bases derived from purine and pyrimidine. The term “nucleic acid” can be used to refer to individual nucleic acid bases or oligonucleotides. These can include, for example, a short chain nucleic acid sequence of at least two nucleotides covalently linked together, typically less than about 500 nucleotides in length, and more typically about 20 to 100 nucleotides in length. The term “nucleic acid” can also refer to long sequences of nucleic acid, such as those found in cDNAs or PCR products, for example, sequences that are hundreds or thousands of nucleotides in length. The exact size of the nucleic acid sequence according to the invention will depend upon many factors, which in turn depend upon the ultimate function or use of the nucleic acid.

Nucleic acids can be prepared using techniques presently available in the art, such as solid support nucleic acid synthesis, DNA replication, reverse transcription, and the like. Alternately, nucleic acids can be isolated from natural sources. The nucleic acid can be in any suitable form, for example, single stranded, double stranded, or as a nucleoprotein. A nucleic acid will generally contain phosphodiester bonds, although, in some cases, a nucleotide can have an analogous backbone, for example, a peptide nucleic acid (PNA). Nucleic acids include deoxyribonucleic acid (DNA) (such as complementary DNA (cDNA)), ribonucleic acid (RNA), and peptide nucleic acid (PNA). The nucleic acid can contain DNA, both genomic and cDNA, RNA or both, wherein the nucleic acid contains any combination of deoxyribo- and ribo-nucleotides. Furthermore, the nucleic acid can include any combination of uracil, adenine, guanine, thymine, cytosine as well as other bases such as inosine, xanthenes, hypoxanthine and other non-standard or artificial bases. PNA is a DNA mimic in which the native sugar phosphate DNA backbone has been replaced by a polypeptide.

As used herein, the terms “complementary” or “complementarity,” when used in reference to nucleic acids (that is, a sequence of nucleotides such as a probe nucleic acid or a target nucleic acid), refer to paired nucleic acid sequences that are able form standard Watson Crick base-pairs. For example, for the sequence “5′-T-G-A-3′,” the complementary sequence is “3′-A-C-T-5′.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

The term “hybridization” is used in reference to the pairing of complementary nucleic acids. Hybridization and the strength of hybridization, that is, the strength of the association between the nucleic acids is influenced by such factors as the degree of complementarity between the nucleic acids, stringency of the conditions involved, the melting temperature (Tm) of the formed hybrid, and the G:C to A:T ratio within the nucleic acids.

Therefore, the array can provide a plurality of nucleic acid probes, each nucleic acid probe defined by having different nucleic acid sequence and separated on the array in individual clusters of microparticles. The nucleic acid probes coupled to the microparticles can be of any length but are preferably at least 8 nucleotides in length. More preferably the nucleic acid probes are between 8 and 200 nucleotides in length. Most preferably the nucleic acids probes are between 12 and 50 nucleotides in length.

In another embodiment the probe can be a protein, or a complex of proteins, and the array includes a plurality of clustered arrangements of microparticles, the microparticles of each individual clustered arrangement coupled to a unique protein probe, or complex of proteins serving as a probe. Each protein, or complex of proteins, has an affinity for a target, which can be present in a sample. The protein probe can be, for example, an antibody, or portions of an antibody, for example, a F_{ab} fragment or F_{ab′} fragments, that specifically recognizes a target or a portion of a target, if present in a sample. The array can therefore comprise a plurality of microparticles coupled to different antibodies, each antibody with a different affinity for a particular target, which may be present in a sample.

In other embodiments, the probe can be a small molecule, for example a molecule that specifically binds to a portion of a protein, a portion such as an enzyme binding pocket or active site. Small molecule probes can include, for example, enzyme inhibitors, enzyme cofactors, enzyme substrates.

As described herein, a particular clustered arrangement of microparticles will be assigned a specific probe, so that the clustered arrangement will be specific for the type of probe coupled to the microparticle. Typically, each microparticle of a particular cluster is coupled with a plurality of identical probe molecules. By providing multiple copies of the same probe molecule on a single microparticle and providing a plurality of microparticles per cluster, the sensitivity of the array can be greatly increased. This can be useful in that, following binding of the target to the probe, a higher signal to noise ratio can be achieved.

The number of probe molecules provided on the microparticles of a cluster can be adjusted by the user to achieve the desired effect. The density of probe molecules, for example, the number of nucleic acid or protein probe molecules coupled to a microparticle can be in the range of 1-260,000 probe molecules per 1 μm diameter microparticle. Typically, 40,000-50,000 probe molecules are immobilized per 1 μm diameter microparticle. However depending on microparticle source and preparation, the amount of probe molecules coupled to the microparticles may vary. For example, if the probe is coupled to the microparticles via a coupling moiety such as streptavidin, the amount of steptavidin on a commercial preparation of streptavidin-coated microparticles can play a factor in the amount of probe that can be coupled to the microsparticles.

According to the invention, probes can be coupled to the microparticles in any suitable manner. For example, the microparticles can be provided with reactive groups on the surface, as described above, which can be used to couple probe molecules. Depending upon the reactive groups selected and the desired probe, the probe may or may not be modified prior to attachment to the microparticle.

In some embodiments, the probe can be modified prior to coupling of the probe with the microparticle. For example, nucleic acids can be derivatized with one member of a binding pair, and the microparticles derivatized with the other member of the binding pair. Suitable binding pairs include, but are not limited to, avidin:biotin, streptavidin:biotin, antibody:hapten, for example, anti-digoxigenin Ab:digoxignenin or anti-trinitrophenyl Ab:trinitrophenyl. For example, the nucleic acid probe can be biotinylated by using enzymatic incorporation of biotinylated nucleotides, or by cross-linking the biotin to the nucleic acid using methods known in the art. A biotinylated nucleic acid probe can then be coupled with streptavidin provided on the surface of the microparticles.

Nucleic acids can be modified in a variety of ways to afford coupling to the microparticles. For example, nucleic acid can be modified to provide a reactive moiety at the 3′ or 5′ end. Alternatively, nucleic acid can be synthesized with a modified base. In addition, modification of the sugar moiety of a nucleotide at positions other than the 3′ and 5′ position is possible through conventional methods. Also, nucleic acid bases can be modified, for example, by using N7- or N9-deazapurine nucleosides or by modification of C-5 of dT with a linker arm, for example, as described in F. Eckstein, ed., “Oligonucleotides and Analogues: A Practical Approach,” IRL Press (1991). Alternatively, backbone-modified nucleic acids, such as phosphoramidate DNA, can be used so that a reactive group can be attached to the nitrogen center provided by the modified phosphate backbone.

Preferably, modification of the probe, for example, a nucleic acid or an antibody, does not substantially impair the ability of the probe to specifically bind to its target. In the case of nucleic acid probe, modification should preferably avoid substantially modifying the functionalities of the nucleic acid that are responsible for Watson-Crick base pairing to the target nucleic acid or, in the case of an antibody, modification should preferably avoid substantially modifying the complementarity-determining regions (CDRs) of the antibody.

A variety of reagents are available for use in modifying nucleic acids. In some embodiments, nucleotides having reactive groups can be synthesized and incorporated into nucleic acids using enzymatic techniques. For example, a variety of reagents are available that can be used to label nucleic acids with biotin, fluorescein and digoxigenin (DIG). A nucleic acid can be labeled with a reactive dideoxyribonucleotides, deoxyribonucleotides, or ribonucleotides, using a terminal transferase, in order to provide either single or multiple reactive groups at the 3′ end of the nucleic acid. For example, a digoxygenin-labeling kit for random primed labeling of DNA with DIG-11-UTP is commercially available (DIG-High Prime; Boehringer-Mannheim) as is a biotin-labeling kit (Biotin High Prime) and a fluorescein-labeling kit (Fluorescein-High-Prime). DNA can also be random-primed with reactive deoxyribonucleotides using the Klenow enzyme.

DNA Polymerase I enzyme can also be used to incorporate reactive nucleotides into a nucleic acid. By including reactive deoxyribonucleotides in the mixture of deoxynucleotide triphosphates (dNTPs), the resulting polymerized product will contain one or more reactive groups along its length. In addition, during polymerase chain reaction (PCR), a reactive deoxyribonucleotide can be included in the mixture of dNTPs for the labeling of amplification products. It is also possible to incorporate a ribonucleotide into RNA, for example, by the use of an RNA polymerase such as SP6 or T7, and standard transcription protocols.

Alternatively, polypeptides, for example proteins, can be passively absorbed onto microparticles and then optionally crosslinked to the microparticles. Polypeptides are preferably absorbed onto the microparticles under conditions that promote the greatest interaction of hydrophobic portions of the polypeptide and the microparticle.

Typically, the probe is coupled to the microparticles prior to disposing of the slurry containing microparticles onto the substrate. The probe can be coupled to the microparticle in a suitable liquid media, such as phosphate buffered saline. Coupling of the probe to the microparticle prior to disposing of the microparticle can provide benefits in array preparation. For example, as compared to coupling probes to a flat substrate, a higher density of probe per surface area of substrate can be achieved by first coupling probes to the microparticles and then disposing the microparticles on a surface. Also, the coupling of probes to microparticles in solution is generally more efficient than the coupling of probes to a “conventional”, flat substrate, resulting in a low loss of probe during the coupling procedure. Additionally, coupling of a probe to a microparticle in solution generally allows for more variability during the coupling process. For example, coupling procedures that require agitation of the coupling solution, such as stirring, can readily be achieved using microparticles in the stirred solution. Additionally, determination of the amount of probe coupled per microparticle can readily be achieved by performing, for example, immunofluorescence flow cytometry or a protein assay, such as a BCA assay, on a portion of the microparticles following coupling to the probe. This can provide greater accuracy in array preparation and assay analysis. Once the microparticles have been coupled with the desired amount and type of probe, these probe-coupled microparticles can then be included in a slurry containing a suitable matrix-forming material.

In a one embodiment, the clustered arrangement of microparticles is immobilized on a substrate within a matrix. As used herein, “immobilization” refers to the process wherein microparticles of a clustered arrangement become positionally fixed within a matrix that is on the surface of a substrate. Typically, immobilization is carried out by mixing microparticles with a matrix-forming material to create a slurry, disposing the slurry on a substrate, and then treating the slurry so the slurry forms a matrix.

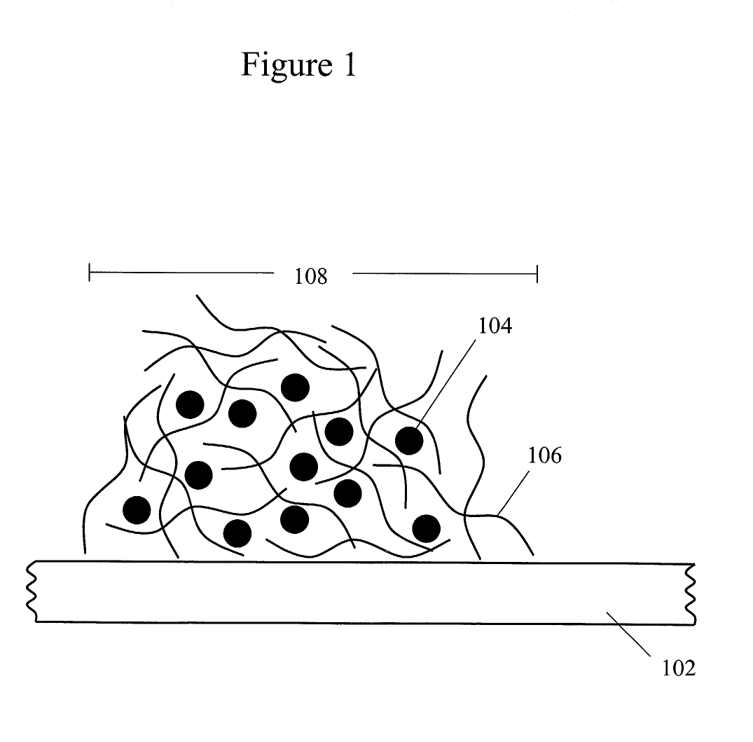

In a preferred embodiment, the microparticles are immobilized on the substrate by entrapment in a polymeric matrix, as defined herein. An example of entrapped microparticles is shown in FIG. 1, wherein a polymeric material 106 is disposed on a substrate 102 and entraps the microparticles 104 within the polymeric material 106 and thereby forms a clustered arrangement of microparticles 108.

The polymeric matrix can be composed of a variety of materials that allows entrapment of the microparticles. Preferred materials for the polymeric matrix can be, but are not limited to, synthetic polymers which include polyacrylamide, polymethacrylamide, polyvinylpyrrolidone, polyacrylic acid, polyethylene glycol, polyvinyl alcohol, and poly(HEMA), copolymers thereof, or any combination of polymers and copolymers. Natural polymers can also be used and include polysaccharides, for example, polydextrans, glycosaminoglycans, for example, hyaluronic acid, and polypeptides, for example, soluble proteins such as albumin and avidin, and combinations of these natural polymers. Combinations of natural and synthetic compounds can also be used. The polymers and copolymers as described can also be derivitized with a reactive group, for example a thermally reactive group or a photoreactive group.

In an alternate embodiment separate crosslinking compounds, for example photoreactive or thermally activated crosslinkers, can be added to the matrix-forming material and can be treated to form the matrix. Addition of compounds such as crosslinkers could serve to make the matrix more durable to use conditions and also can create matrices with smaller pore sizes capable of entrapping smaller microparticles.

The polymeric matrix is typically formed by providing an external stimulus to bind the polymer to the substrate and crosslink the polymer. In one embodiment, a slurry including microparticles and polymer is disposed on the substrate and then the polymer is treated to crosslink the polymer, for example, by activation of reactive groups provided by the polymer.

In some embodiments the reactive groups provided on the polymer can be photoreactive groups and the photoreactive polymer can be crosslinked by irradiation. The microparticles become entrapped in the polymeric matrix which is formed by crosslinking of the polymers. The photoactive groups can also serve to bind the polymer to the surface of the substrate upon activation of the photoreactive groups. Different concentrations of polymer can be present in the slurry but generally the concentration shouts be great enough to allow for entrapment of the microparticles. The concentration of the polymer can also depend on the size of the microparticles used. For example, the concentration of polymer is at least 0.625 mg/mL for a 1.0 μm microparticle in order to stably entrap the microparticle in the polymeric matrix.

According to this embodiment, photoreactive groups can be provided on a polymer. As used herein, a “photoreactive polymer” can include one or more “photoreactive groups.” A “photoreactive group” includes one or more reactive moieties that respond to a specific applied external energy source, such as radiation, to undergo active species generation, for example, active species such as nitrenes, carbenes and excited ketone states, with resultant covalent bonding to an adjacent targeted chemical structure. Examples of such photoreactive groups are described in U.S. Pat. No. 5,002,582 (Guire et al., commonly owned by the assignee of the present invention), the disclosure of which is incorporated herein in its entirety. Photoreactive groups can be chosen to be responsive to various portions of the electromagnetic spectrum, typically ultraviolet, visible or infrared portions of the spectrum. “Irradiation” refers to the application of electromagnetic radiation to a surface.

The photoreactive groups can be located at any position on the polymer. Preferably, the number of photoreactive groups present on the polymers is increased when smaller microparticles are to be immobilized.

Photoreactive aryl ketones are preferred photoreactive groups on the photoreactive polymer, and can be, for example, acetophenone, benzophenone, anthraquinone, anthrone, and anthrone-like heterocycles (i.e., heterocyclic analogs of anthrone such as those having N, O, or S in the 10-position), or their substituted (e.g., ring substituted) derivatives. Examples of preferred aryl ketones include heterocyclic derivatives of anthrone, including acridone, xanthone and thioxanthone, and their ring substituted derivatives. Particularly preferred are thioxanthone, and its derivatives, having excitation wavelengths greater than about 360 nm.

The azides are also a suitable class of photoreactive groups on the photoreactive polymer and include arylazides (C₆R₅N₃) such as phenyl azide and particularly 4-fluoro-3-nitrophenyl azide, acyl azides (—CO—N₃) such as ethyl azidoformate, phenyl azidoformate, sulfonyl azides (—SO₂—N₃) such as benzensulfonyl azide, and phosphoryl azides (RO)₂PON₃ such as diphenyl phosphoryl azide and diethyl phosphoryl azide.

Diazo compounds constitute another suitable class of photoreactive groups on the photoreactive polymers and include diazoalkanes (—CHN₂) such as diazomethane and diphenyldiazomethane, diazoketones (—CO—CHN₂) such as diazoacetophenone and 1-trifluoromethyl-1-diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as 3-trifluoromethyl-3-phenyldiazirine, and ketenes (—CH═C═O) such as ketene and diphenylketene.

Exemplary photoreactive groups are shown as follows.

TABLE 1

| Photoreactive Group | Bond Formed |
|---|---|
| aryl azides | Amine |
| acyl azides | Amide |
| Azidoformates | Carbamate |
| sulfonyl azides | Sulfonamide |
| phosphoryl azides | phosphoramide |
| Diazoalkanes | new C—C bond |
| Diazoketones | new C—C bond and ketone |
| Diazoacetates | new C—C bond and ester |
| beta-keto-alpha-diazoacetates | new C—C bond and beta-ketoester |
| aliphatic azo | new C—C bond |
| Diazirines | new C—C bond |
| Ketenes | new C—C bond |
| photoactivated ketones | new C—C bond and alcohol |

The photoreactive polymer can, in some embodiments, comprise a photoreactive copolymer. The polymer or copolymer can have, for example, a polyacrylamide backbone or be a polyethylene oxide-based polymer or copolymer. One example of a photoreactive polymer comprises a copolymer of vinylpyrrolidone and N-[3-(4-Benzoylbenzamido)propyl]methacrylamide (BBA-APMA); another example is a copolymer of acrylamide and BBA-APMA.

The photoreactive groups of the photoreactive polymer can allow the formation of a covalent bond between the substrate and the photoreactive polymer thereby binding the polymer to the surface of the substrate. The photoreactive groups of the photoreactive polymer can also serve to crosslink to proximal polymeric strands together, allowing the formation of a network of covalently crosslinked polymeric strands that serve as the matrix in which the microparticles can be entrapped within. In some embodiments, a non-photoreactive crosslinking agent can be used to promote the formation of crosslinked polymeric strands. Use of a crosslinking reagent, for example, bis-acrylamide, can depend on the location and number of photoreactive groups that are present on the polymeric strand.

The polymeric matrix can be composed of a variety of materials that preferably have pore sizes which allow the entrapment of the microparticle of the invention. Preferably the matrix does not allow the microparticles to escape from the porous material during steps involving detection of a target in a sample. For example, if entrapping microparticle with an average diameter of 2.5 μm, it is useful to have a pore size in the range of 50 nm to 2.5 μm, and more preferably in the range of 100 nm to 1 μm.

In another embodiment, immobilization of the microparticles can be performed by chemical bonding of the microparticle to the polymeric matrix and the polymeric matrix to the substrate. A variety of bonds can be formed between the microparticles and the matrix material, and the matrix material to the substrate, which allows immobilization of the microparticle. These bonds include, for example, ionic, covalent, cooordinative, hydrogen or Van der Waals bonds. In this embodiment, covalent bonds are preferably formed.

According to the invention, arrays are fabricated by providing a substrate, preparing a slurry containing matrix-forming material and probe-coupled microparticles, disposing the slurry on the substrate in a manner to form clustered arrangements of microparticles, and treating the slurry to form a matrix, thereby immobilizing the microparticles in the matrix.

In one embodiment, slurries including matrix-forming material and probe-coupled microparticles are printed onto the surface of the substrate to form an array. According to this embodiment, printing devices physically spot the slurry onto the substrate surface and are available from a variety of sources, including BioRobotics Ltd. (MicroGrid arraying robot, Comberton, Cambridge, UK) and Packard Instrument Company (BioChip arrayer, Meriden, Conn.). Alternatively, the probe can be applied jet printed to the microparticles through utilization of a piezoelectric pump. The BioChip Arrayer (Packard BioChip Technologies) can be used to print the slurries onto spots of the substrate.

When printing or spotting techniques are used to apply the probe to the microparticles, typically a volume of approximately 0.5 to 1.0 nL of solution containing the probe molecules is applied at each microparticle. Preferably, the volumes in the range of 1 pL to 5 μL. When contact printing is used, the volume of slurry, containing the microparticles, that is applied to the substrate will depend upon such factors as, for example, the size of the microparticles used in the array, the desired amount of probe provided on the substrate, the type of printing pin utilized, the surface energy of the substrate, the surface tension of the microparticle-containing slurry solution, for example, the probe molecules in solvent, and the like. When inkjet printing techniques are used, characteristics of the substrate will not typically determine the volume of solution containing probe solution applied. Typically, piezoelectric printing will involve application of approximately 10-100 pL of slurry.

The clustered arrangement of microparticles can cover at least a portion of the surface of the substrate. The clustered arrangements of microparticles can be patterned at various locations on the surface of the substrate. Each clustered arrangement of microparticles typically is coupled to a unique probe, therefore the location of probes on the surface of the substrate is determined by the location or pattern of the clusters on the substrate. The thickness of the polymeric matrix of each cluster can vary and can depend on the size of the microparticles entrapped in the polymeric matrix. Preferably, the thickness of the polymeric matrix on the substrate is greater than the diameter of the largest microparticle being disposed on the substrate.

A target can be a molecule, a cell, or a particle suspected to be present in a sample that can specifically interact with a particular probe, based on interactions exemplified above. The target can be detected and/or quantitated, according to the methods described herein and can be coupled to a “target marker” to accomplish this. The target can comprise naturally occurring or man-made molecules, and it can be used in its unaltered state or as aggregates with other species. Examples of targets include antibodies, nucleic acids, receptors, hormones, drugs, metabolites, cofactors, peptides, enzymes, viral particles, cells and the like. In one embodiment, the target comprises a nucleic acid to be detected in a sample. The probe and target are typically members of a specific binding pair, wherein the members of the pair are known to bind to each other, while binding little or not at all to other nonspecific substances.

In one embodiment, the sample suspected to contain a target is treated to label the target with a target marker. As used herein, “target marker”, refers to the detection moiety that is used to visualize the target. As contemplated in this invention, a target marker comprises a moiety that is detectable using standard techniques known in the art. Examples of suitable markers include, but are not limited to, fluorophores, phosphors, and radioisotopes.

When the target to be detected comprises RNA, the RNA target can be labeled using molecular biology techniques, such as in vitro run-off transcription to generate a labeled RNA sample using RNA polymerases, for example T7, T3, or SP6 RNA polymerases. Kits for labeling RNA are available from various sources, for example, Ambion, Inc. (Austin, Tex.). This technique can be particularly useful in generating labeled RNA from, for example, a cDNA library that has been cloned into a vector with the appropriate promoters for RNA polymerase transcription. Techniques can also be used to generate labeled DNA, for example, nick translation, PCR amplification, random priming, or primer extension. These techniques can be useful for generating labeled DNA from for example, cDNA libraries or genomic DNA libraries. Modified DNA nucleotides for use as labels can also be created from Reverse Transcriptase reactions. For example, an RNA sample, such as a polyA-RNA sample can be used as a template in a reaction containing Reverse Transcriptase, polyT oligonucleotide primer, and modified nucleotide to generate labeled-cDNA. Techniques for labeling DNA can be found in various technical references, for example, Current Protocols in Molecular Biology (Ausubel et al., ed., 1990, Greene Pub. Associates and Wiley-Interscience: John Wiley, New York).

RNA and DNA targets can be labeled using modified nucleotides, for example fluorophore-coupled nucleotides, such as Fluorescein-5[6]-carboxyamidocaproyl-[5-(3-aminoallyl)uridine 5′triphosphate (Sigma, St. Louis, Mo.), biotin-coupled nucleotides, such as (N⁶[N-(Biotyinyl-ε-aminocaproyl)-6-aminohexylcarbamoylmethyl]adenosine 5′-triphosphate) or other modified nucleotides, for example, 5-(3-aminoallyl)uridine 5′-triphosphate (Sigma, St. Louis, Mo.) in order to enable detection of the DNA or RNA. Secondary fluorophore-coupled reagents, for example, Streptavidin-Cy3 (Caltag, Burlingame, Calif.) can be used to for indirect detection of the modified nucleic acid. Radioactive nucleotides, for example ³²P-, ³³P-, and ³⁵S-labeled ribonucleotides and deoxyribonucleotides can be incorporated into the target DNA or RNA present in a sample. These modified nucleotides can also be used to label sample nucleic acids in other ways, for example, by 5′ or 3′ end-labeling with enzymes such as polynucleotide kinase or terminal transferase. Optionally, kits and instructions for coupling modified nucleotides to nucleic acid samples can be obtained commercially from, for example, CALBIOCHEM (San Diego, Calif.). Labeled target can optionally be purified by methods such as gel filtration or purification, spin columns, or selective precipitation.

In another embodiment, the sample includes a plurality of protein suspected of containing a protein target. The protein sample can be obtained from a tissue sample, such as a biopsy, or from a fluid sample containing cells, for example, blood or bone marrow, or from other body fluids, for example, plasma, sweat, saliva, or urine. A protein sample can be recovered from body fluid by a variety of techniques, for example, by precipitation, filtration, or dialysis. A protein sample from cells, for tissue or body fluid, can be prepared by the lysis or solubilization of cells in detergents, optionally using methods such as sonication or homogenization. Ionic or non-ionic detergents can be used, for example, sodium dodecyl sulphate (SDS), Triton X-100, sodium deoxycholate and CHAPS. Cells can also be disrupted in the presence of chaotropic reagents, such as urea and guanidine salts. Other reagents can be added to the detergent or chaotropic reagent, such as a buffer, for example, Tris or HEPES, and salts, for example, KCl or NaCl. Other compounds can be utilized which stabilize the protein sample, for example, protease inhibitors, such as PMSF, pepstatin, or EDTA. However, a variety of methods and buffer compositions are available for the lysis or disruption of cells for protein extraction and are commonly known in the art. This information can be found in various references, for example, Current Protocols in Protein Science (Coligan et al., eds., 1996, John Wiley & Sons, New York, N.Y.).

The protein sample is preferably labeled in such a way to enable detection of the protein target and to retain the ability of the protein target to interact with the probe coupled to the microparticle. Reagents are available that allow the coupling of a fluorophore to an amino acid residue on a protein target. Amine-reactive groups, for example, succinimidyl esters, including sulfosuccinimidyl esters, isothiocyanates and sulfonyl chlorides, or dichlorotriazines, aryl halides and acyl azides are available as fluorophore probes and can be used for protein target labeling. A variety of these amine-reactive fluorophore probes are commercially available, for example, Alexa Fluor™ 350 carboxylic acid, succinimidyl ester; 4,4-difluoro-5-(2-thienyl)-4-bora-3a,4a-diaza-s-indacene-3-propionic acid, succinimidyl ester (BODIPY™ 558/568, SE); and 6-carboxy-4′,5′-dichloro-2′, 7′-dimethoxyfluorescein, succinimidyl ester (6-JOE, SE) (Molecular Probes, Eugene, Oreg.). In other circumstances it can be desirable to label the protein target with a fluorescent thiol-reactive derivative, for example, N,N′-didansyl-L-cystine (Molecular Probes, Eugene, Oreg.). Alternatively, other reagents, for example fluorescent dyes containing a hydrazine group, an aromatic diazonium salt, or an amine group can be used to label the protein sample.

The protein target can also be coupled to a fluorescent protein, for example the Green Fluorescent Protein (GFP), using commercially available bifunctional crosslinking reagents, for example NHS-ASA (Pierce Chemical, Rockford, Ill.). Other bifunctional crosslinking agents that are reactive toward amine, sulfhydryl, carbohydrate, carboxyl and hydroxyl groups are commercially available (for example, Pierce Chemical, Rockford, Ill.) and can be used for coupling a protein of interest to the protein target. The protein target can also be coupled to a primary reagent for secondary fluorophore detection. In some embodiments, the protein can be modified by, for example, sulfo-NHS-biotin, and then coupled to a secondary fluorophore reagent, for example Streptavidin-Cy3.

Other methods of labeling a protein sample for detection are available. Such methods include, for example, protein iodination using ¹²⁵I and protein phosphorylation using ³²P or ³³P and a protein kinase can be used.

In some instances, the target includes a particle, for example, a viral particle, a cell, or a portion of a cell. These targets can display molecules on their surfaces that allow them to bind a particular probe that can be present in the array. The binding of the probe and the molecule present on a viral particle, cell, or portion of a cell can be useful in identifying and quantifying a particular viral particle, cell, or portion of a cell which can be present in a sample. For example, the array can be useful in determining the presence and quantity of different subtypes of lymphocytes present in a sample. The viral particle, cell, or a portion of a cell can be labeled by a variety of means, for example, by labeling with an antibody that is coupled to a fluorophore. The labeled antibody can be chosen to react specifically with a subpopulation of the sample or non-specifically with the sample. Alternatively, the viral particle, cell, or a portion of a cell can be labeled by incorporation of a fluorescent dye into the membrane of these potential targets. Such dyes, for example, PKH-67 GL (Sigma, St. Louis, Mo.) or a fluorescent lipophilic probe, CM-DiI (Molecular Probes, Eugene, Oreg.) are commercially available and can be used to label cells.

It is understood by one of skill in the art that there are a wide variety of techniques available for protein labeling and that the technique chosen can depend on the protein or proteins available in the sample targeted for labeling.

In an alternate embodiment, target detection can be performed by employing a fluorophore:quencher pair on the microparticle. In this embodiment, probe-coupled microparticles are further coupled to a detectable moiety, such as a fluorescent molecule, and target is coupled to a compound that quenches the signal of the detectable moiety on the microparticle. Binding of the quencher-coupled target to the probe on the fluorophore-coupled microparticle will result in the loss of signal from the clustered arrangement of microparticles. For example, in clustered arrangements of probe- and fluorescein-coupled microparticles, a target, specific for the probe and coupled to the quenching molecule 6-(N-4′-carboxy-4-(dimethylamino)azobenzene)-aminohexyl-1-O-(2-cyanoethyl)-(N,N-diisopropyl)-phosphoramidite (Dabeyl; Glen Research, Sterling, Va.) can specifically bind probe and effectively quench the fluorescence emission of fluorescein. Quenching of the fluorescent signal can be accomplished by positioning the quenching molecule proximal to the fluorophore.

Given the description herein, one of skill in the art can select the desired labeling scheme depending upon the target to be detected. While nucleic acids and proteins have been described with particularity, it will be clear that the teaching herein can be applied to label samples suspected to contain other types of targets, including, but not limited to, small molecules and the like.

Once formed, the array, which can contain a plurality of clustered arrangements of microparticles, each clustered arrangement of micropsheres associated with a unique probe, can be used to detect target suspected to be contained in a sample. In use, a sample is modified to provide labeled target, as described herein. The modified sample is then applied to the array, and the array and sample are maintained under conditions suitable for specific binding of the target, if present in the sample, to the probe. Such specific binding conditions can be determined using techniques known in the art, depending upon the target to be detected. For example, when nucleic acid is used as the probe, specific binding, or hybridization conditions can be adjusted based on the number of nucleotide base-pairs that are formed between the probe nucleic acid and the target nucleic acid.

After specific binding of the target to the probe, excess sample can be removed, for example, by washing, and the remaining hybridized targets can be interrogated. The arrays of the invention can be used to detect any target suspected to be present in a sample. Interrogation of a sample can involve one or more visualization steps depending on the types of markers used for the target detection (target marker) and optionally microparticle detection if there is any microparticle probe included in the array.

Determination of the presence and amount of one or more target(s) in a sample is typically performed by visualizing the presence or absence of a detectable signal associated with one or more clustered arrangements of microparticles on a substrate. By knowing the probe associated with a particular clustered arrangement of microparticles on the substrate, and the signal associated with a particular clustered arrangement of microparticles, one can determine the presence and amount of particular targets in a sample. Visualization of the detectable markers can be accomplished using any suitable visualization technique known in the art. Fluorescence imaging can be made using a modified epifluorescence microscope or a fluorescence confocal microscope. Suitable microscopes include, for example, an Olympus BX60 (Tokyo, Japan) or other similar microscopes. Fluorescence images from microscopy images can be analyzed for fluorescence intensity using computer software. Commercially available microscopy analysis software, for example, Image-Pro Plus (version 4.0) (Media Cybernetics, L.P., Silver Spring, Md.), can be used to define and count fluorescent signals automatically with optical detection systems. Alternatively, the method of fluorescence scanning can be used to visualize particles. Fluorescence scanners such as the Scan Array 5000 (GSI Lumonics, Billerica, Mass.) or Axon GenePix 4000A (Foster City, Calif.), which have resolution of approximately 5 μm, can be used for visualization.

### EXAMPLES

### Example 1

As shown in this example, an array was fabricated by disposing slurries containing probe-coupled microparticles in a matrix-forming material. Microparticles were prepared by coupling a probe to the microparticle, followed by preparation of a slurry containing a photoreactive polymer and the probe-coupled microparticles. These prepared microparticles were then applied to a substrate to form an array.

Magnetic, polystyrene-encapsulated microparticles, coupled to a fluorescent blue dye (excitation/emission maxima of 490/515 nm) and streptavidin were obtained from Bangs Laboratories (Product #CM01F; Fishers, Ind.). The streptavidin concentration was measured by biotin-conjugate binding and was determined to be 9.86 μg biotin-alkaline phosphatase/mg microbeads and 0.77 μg biotin-FITC/mg microbeads per manufacturer's measurement. The microparticles had a diameter of 0.96 μm and a density of 1.7 g/cm³ (1.305e+12 microparticles/g). The microparticles, were washed twice with deionized water and resuspended in 25 mM phosphate buffered saline, pH 8, at 10 mg/mL.

The streptavidin-coated microparticles were coupled to oligonucleotide BN30. BN30 is a 30-mer with a 5′ biotin modification and a 3′ amine (Integrated DNA Technologies, Coralville, IA). Coupling was performed using 5 nmole/ml of BN30 and 1 mg/ml of microparticles (five-fold excess of biotinylated oligonucleotide, based upon the supplier's stated biotin binding capacity) in 25 mM PBS, pH 8. The coated microparticles and oligonucleotide were incubated for 30 minutes at room temperature with gentle agitation. After incubation, the microparticles were washed with deionized water and resuspended at 20 mg/ml in deionized water.

A slurry of 37 mg/ml photoreactive poly(vinylpyrrolidone) (PV01; SurModics, Inc., Eden Prairie, Minn.) in water was combined with the microparticle solution at a ratio of 9:1 (10×dilution of microparticle solution in PVO1). The slurry was printed onto an acrylic surface (obtained from Cadillac Plastics, Minneapolis, Minn.) using a Microgrid II arrayer (Biorobotics, Inc. Cambridge, UK) with an average spot size of 100 μm and a center to center spacing of 250 μm, providing approximately 16 spots/mm². The coated slides were air dried and irradiated for two (2) minutes with broad spectrum ultraviolet light (320-390 nm) using a Dymax LightWelder PC-2 (Dymax Engineering Adhesives, Torrington, Conn.) having a typical power output of 2 mW/cm². The lamp was positioned approximately 10 cm from the slides which were also placed beneath a cut-off filter (315 nm) to avoid potential nucleic acid damage, while gelling the PV01 solution. After irradiation, the substrate was rinsed with 1×PBS, 0.1% Tween 20.

The arrays prepared by the above method were stable to washing and touching, while samples that were not irradiated were not stable. Fluorescence scanning and light microscopy were used to determine the presence and location of the microparticles Sample containing target nucleic acid was applied to the array and incubated. 2.5 μL of a 33 fM solution of a fluorophore-coupled target nucleic acid in hybridization buffer (5×SSC, 0.1% SDS, 0.1 mg/ml salmon sperm DNA) per cm² (array area) placed between a coverslip and the array surface. The slides were then be placed in hybridization chambers and heated in a water bath at 45° C. for 2 hours. The coverslips were then removed with a stream of 4×SSC buffer and the slides were then washed with 2×SSC/0.1% SDS for five minutes at 45° C., followed by a 0.2×SSC wash for one minute at room temperature, and finally a wash of 0.1×SSC for one minute at room temperature. The slides were then spun dry and the target oligonucleotide detected by methods described below.

Target was detected on the array with a ScanArray 5000 fluorescence scanner (Packard Bioscience, Billerica, Mass.).

### Example 2

An array was formed by entrapping microparticles in a polymeric matrix. Entrapment of the microparticles in the polymeric matrix did not depend on the formation of covalent or ionic bonds between the substrate and the microparticles. In this example, matrices formed from poly(vinyl pyrrolidone)(PVP) were used to entrap microparticles. An array was fabricated by preparing a mixture of photoreactive PVP and underivatized silica microparticles, disposing the mixture on a substrate, and then polymerizing the photoreactive PVP to a PVP matrix.

Silanated glass slides (1×3 in.×1 mm) were used in the preparation of substrates in array fabrication. Glass microscope slides were obtained from Erie Scientific, Portsmith, N. H. (catalog #2950-W). These soda lime glass microscope slides were silane treated by dipping in a mixture of 1% v/v p-tolyldimethylchlorosilane (T-Silane) and 1% v/v n-decyldimethylchlorosilane (D-Silane, United Chemical Technologies, Bristol, Pa.) in acetone for 1 minute. After air drying, the slides were cured in an oven at 120° C. for one hour. The slides were then washed with acetone followed by DI water dipping. The slides were further dried in an oven for 5-10 minutes. The silanated glass or polypropylene slides were then washed in acetone or isopropanol.

A solution of 10 mg/ml photoreactive poly(vinylpyrrolidone) (PV01; SurModics, Inc. Eden Prairie, Minn.) and 2.5 mg/ml of 5 μm-diameter silica microparticles (Product #SSO5N, Bangs Laboratories, Fisher, Ind.) in deionized water was printed using 25 gauge disposable needles (PrecisionGlide Needles, Becton Dickinson and Co., Franklin Lakes, N.J.) and an x-y programmable stage a glass microscope slide as prepared above. The slide was then irradiated for two minutes with broadband ultraviolet light over the range of 320-390 nm (Dymax Engineering Adhesives, Dymax LightWelder PC-2, Torrington, Conn.). Following this, the slide was rinsed five times with 1×PBS with 0.05% Tween-20, then incubated for 15 minutes at 45° C. in a blocking solution DB02 (50 mM ethanolamine, 0.1M Tris, pH 9; SurModics, Inc., Eden Prairie, Minn.) supplemented with 0.1% sodium dodecyl sulphate (SDS). The slide was further rinsed with deionized water and incubated for one hour at 45° C. in 4×SSC (0.6 M NaCl, 0.06 M NaCitrate/0.1% SDS. Final rinses included five minutes with 2×SSC at 45° C., then one minute in 0.2×SSC at room temperature, then one minute in 0.1×SSC at room temperature followed by drying.

The microparticles remained inside the PVP hydrogel array and were visualized by fluorescence microscopy (Olympus BX60, Tokyo, Japan) and with a confocal fluorescence scanner (ScanArray 5000, GSI Lumonics, Billerica, Mass.).

When the microparticles and photoreactive PVP were deposited on the glass slide without the step of irradiation, the microparticles were not retained in the gel. The absence of radiation did not allow crosslinks to form in the polymeric matrix, thereby not allowing the polymeric matrix to physically entrap the microparticles.

In order to define the nature of immobilization of microparticles in the polymeric matrix, microparticles 5 μm diameter (Cat no. SS05N, Bangs Laboratories, Fisher, Ind.) were irradiated in a solution of benzophenone dimers functionalized with quaternary amine groups. A solution of PR03 (ethylene(4-benzoylbenzyldimethylammonium)dibromide; SurModics, Inc. Eden Prairie, MN) is described in U.S. Pat. No. 5,714,360 (Swan et al., issued Feb. 3, 1998, commonly owned by the assignee of the present invention, the disclosure of which is incorporated herein in its entirety), at a concentration of 5 mg/ml containing 2.5 mg/ml 5 μm-diameter silica microparticles (Product #SSO5N, Bangs Laboratories, Fisher, Ind.) in 10% v/v DMF/water was printed using 25 gauge disposable needles (PrecisionGlide Needles, Becton Dickinson and Co., Franklin Lakes, N.J.) and an x-y programmable stage onto a hydrophobic glass slide, as performed in Example 1. The printed array was irradiated for two minutes with ultraviolet light (Dymax Engineering Adhesives, Dymax Light Welder PC-2, Torrington, Conn.) and rinsed with deionized water, with 1×PBS with 0.05% Tween-20 added, then incubated for 15 minutes at 45° C. in DB02 (SurModics, Inc., Eden Prairie, Minn.) with 0.1% SDS added. The slide was then rinsed with deionized water and further incubated for one hour at 45° C. in 4×SSC/0.1% SDS. Final rinses included five minutes with 2×SSC at 45° C., then one minute in 0.2×SSC at room temperature, then one minute in 0.1×SSC at room temperature followed by drying.

When imaged at this point, no microparticles remained in the areas printed with the PR03 solution. High concentrations of PR03 proved to be insufficient for immobilization of the microparticles, as opposed to the PV01 polymer, which efficiently trapped the microparticles.

### Example 3

Microparticles were immobilized on the surface of a substrate by entrapment of the microparticles in a polymeric matrix. This method of immobilization does not interfere with the surface functionality of the microparticle since no chemical bonds were formed between the microparticle and polymeric matrix. Therefore, the surface chemistry on the microparticle is not altered. Covalent bonds between polymer molecules were formed after a slurry containing microparticle and polymeric material was coated on the device. Covalent bonds were not formed between the polymer and the microparticle since the microparticles do not present carbon-hydrogen bonds of which the activated benzophenone can react with. Irradiation of the photopolymer crosslinked the polymer with itself encasing the microparticles, but did not alter the biomolecules attached to the microparticles.

To demonstrate this immobilization method, plain silica microparticles of four different diameters (0.4 μm, 0.9 μm, 5.0 μm, and 9.9 μm) were immobilized in photopolymer matrices of differing concentrations. At the lowest concentration of photopolymer the polymeric matrix formed was not sufficient to physically immobilize the microparticles of the sizes tested. As a additional control, some photopolymer coatings were not irradiated which resulted in insufficient crosslinking around the microparticle and leading to microparticle loss from the coating. Stringent rinses in detergent and high salt solutions at elevated temperatures were used to ensure that the photopolymer coatings were robust. 20 μl of 100 mg/ml aliquots of each 400 nm, 970 nm, 5 μm, and 9.9 μm silica microparticles solutions (cat. #SS02N, SS03N, SS05N, and SS06N, respectively; Bangs Laboratories, Fisher, Ind.) were pelleted by centrifugation. These pellets were individually resuspended in 100 μl serial dilutions of photoreactive poly(vinylpyrrolidone) (PV01; SurModics, Inc, Eden Prairie, Minn.). The dilution series consisted of 10, 5, 2.5, 1.25, 0.6, 0.3, 0.15, 0.08, 0.04 and 0.02 mg/ml concentration samples of PV01. The final concentration of microparticles was 20 mg/ml for all sizes. Therefore, the 400 nm microparticle solution contained significantly more microparticles than did the 9.9 μm microparticle solution; however all microparticle solutions contained the same percent solids.

Glass microscope slides, as described in Example 1, that had been coated with a 1% v/v solution of n-decyldimethylchlorosilane and tolyldimethylchlorosilane in acetone were used as a substrate for coating. 5 μl of each microparticle-PV01 solution was placed in an area of approximately 10 mm×2 mm on a glass slide and allowed to air dry. Once dry, the coatings were irradiated with an ultraviolet lamp as detailed in Example 1 (Dymax Lightwelder PC-2, Dymax Engineering Adhesives, Torrington, Conn.) for two minutes. A second set of samples was not irradiated to serve as a control to determine whether crosslinking the photopolymer polymeric matrix was necessary to contain the microparticles.

To determine if the microparticles were entrapped well in the polymer coating, the coated slides were washed in a IX PBS-0.1% Tween-20 solution with mild shaking for one hour, followed by two deionized water rinses. The coatings were then re-examined by microscope and the microparticle loss evaluated. A second wash condition of higher salt with higher temperature was then conducted. The microparticle coated pieces were incubated and shaken in 4×SSC buffer with 0.1% SDS for 45 minutes at 45° C., then rinsed twice with deionized water and examined microscopically for changes in the microparticle coating. Finally a longer high salt wash step was conducted, with the slides incubating in 5×SSC buffer with 0.1% SDS for two hours at 45° C., followed by four rinses of decreasing concentration of SSC.

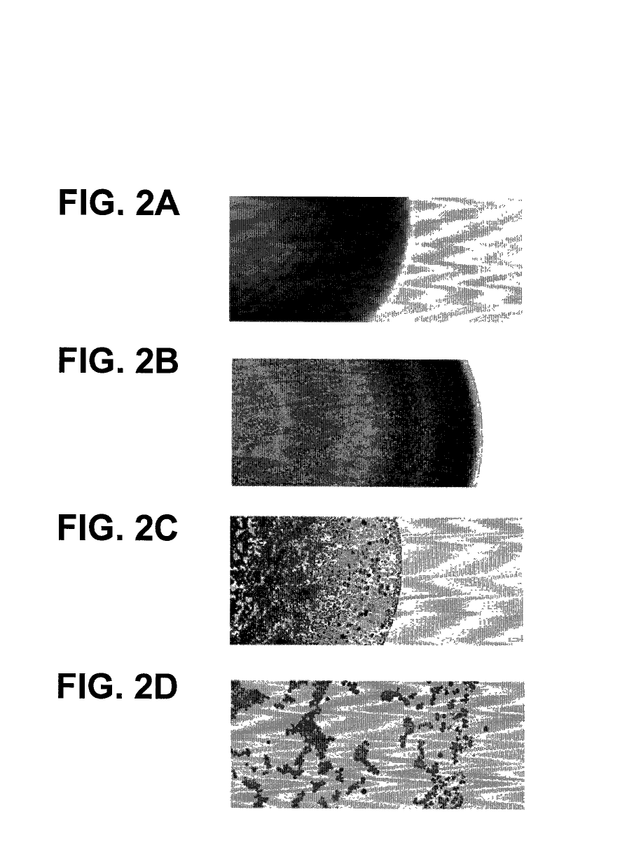

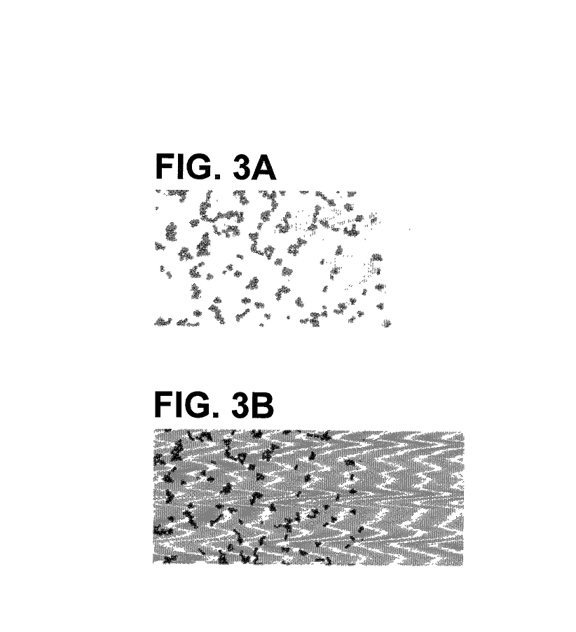

The microparticle coatings were examined by qualitative microscopic examination at 50× magnification (Olympus BX60, Tokyo, Japan) and the changes were tabulated. Results are shown in FIG. 2 and FIG. 3.

FIGS. 2a-2d shows presence of the microparticles after the final wash. FIG. 2a shows a coating having 400 nm microparticles; FIG. 2b shows a coating having 970 nm microparticles; FIG. 2c shows a coating having 5 μm nm microparticles; and FIG. 2d shows a coating having 9.9 μm microparticles.

A micrograph of microparticles before and after all three washes is show in FIG. 3. FIG. 3a shows 9.9 μm microparticles in a 0.3 mg/ml PV01 matrix before washing and FIG. 3b shows 9.9 μm microparticles in a 0.3 mg/ml PV01 matrix after washing. The results are shown in Table 2 and Table 3. As indicated in Tables 1 & 2: N=No Loss, S=Slight Loss, M=Moderate Loss, H=Heavy Loss, C=Complete Loss; PBST=1×PBS-0.1% Tween-20 wash for one hour at room temperature; 4×SSC=4×SSC-0.1% SDS wash for 45 minutes at 45° C.; Final=5×SSC-0.1% SDS wash for two hours at 45° C.

TABLE 2
Irradiated

| PV01 | | | | | | | | | | | | | | | |
|---|---|---|---|---|---|---|---|---|---|---|---|---|---|---|---|
| mg/ml | 10 | | | 5 | | | 2.5 | | | 1.25 | | | 0.6 | | |
| Wash | PBST | 4XSSC | Final | PBST | 4XSSC | Final | PBST | 4XSSC | Final | PBST | 4XSSC | Final | PBST | 4XSSC | Final |
| Step | | | | | | | | | | | | | | | |
| 400 nm | N | N | N | N | N | N | N | N | N | N | N | N | N | N | N |
| 970 nm | N | N | N | N | N | N | N | N | N | N | N | N | N | N | N |
| 5 μm | N | N | N | N | N | N | N | N | N | N | N | N | N | N | N |
| 9.9 μm | N | N | N | N | N | N | N | N | N | N | N | N | N | N | N |
| PV01 | | | | | | | | | | | | | | | |
| mg/ml | 0.3 | | | 0.15 | | | 0.08 | | | 0.04 | | | 0.02 | | |
| Wash | PBST | 4XSSC | Final | PBST | 4XSSC | Final | PBST | 4XSSC | Final | PBST | 4XSSC | Final | PBST | 4XSSC | Final |
| Step | | | | | | | | | | | | | | | |
| 400 nm | N | N | S | S | S | S | S | S | S | M | H | H | H | C | |
| 970 nm | N | N | S | S | S | S | S | S | S | H | C | | H | C | |
| 5 μm | N | N | N | S | S | S | S | M | M | M | H | H | M | H | H |
| 9.9 μm | N | N | N | S | S | S | S | M | M | M | H | H | M | H | H |

TABLE 3
Non-Irradiated

| PV01 | | | | | | | | | | | | | | | |
|---|---|---|---|---|---|---|---|---|---|---|---|---|---|---|---|
| mg/ml | 10 | | | 5 | | | 2.5 | | | 1.25 | | | 0.6 | | |
| Wash | PBST | 4XSSC | Final | PBST | 4XSSC | Final | PBST | 4XSSC | Final | PBST | 4XSSC | Final | PBST | 4XSSC | Final |
| Step | | | | | | | | | | | | | | | |
| 400 nm | M | C | | M | C | | M | C | | M | C | | M | C | |
| 970 nm | M | C | | M | C | | M | C | | M | C | | M | C | |
| 5 μm | M | C | | M | C | | M | C | | M | C | | M | C | |
| 9.9 μm | M | C | | M | C | | M | C | | M | C | | M | C | |
| PV01 | | | | | | | | | | | | | | | |
| mg/ml | 0.3 | | | 0.15 | | | 0.08 | | | 0.04 | | | 0.02 | | |
| Wash | PBST | 4XSSC | Final | PBST | 4XSSC | Final | PBST | 4XSSC | Final | PBST | 4XSSC | Final | PBST | 4XSSC | Final |
| Step | | | | | | | | | | | | | | | |
| 400 nm | M | C | | C | | | H | C | | C | | | C | | |
| 970 nm | M | C | | C | | | H | C | | H | C | | C | | |
| 5 μm | M | C | | C | | | M | C | | M | C | | C | | |
| 9.9 μm | M | C | | H | C | | H | C | | H | C | | H | C | |

As shown above in Tables 2 and 3, microparticle loss was significantly increased in samples that had not been irradiated. Also shown above in Tables 2 and 3, microparticle loss was increased when the concentration of polymer was reduced. Microparticle loss was increased when a smaller size of microparticle was used in the polymeric matrix. This data indicates that irradiation promoted the formation of an encasing polymeric network around the microparticles. This data also indicates that the microparticles were immobilized by physical constraints of the polymeric matrix rather than by chemical bonding.

When printed on a substrate the slurries having smaller microparticles gave coatings that densely packed with multiple layers of microparticles, whereas slurries with larger microparticles gave coatings had loosely arranged microparticles every few tenths of a millimeter. Coatings showing the lower density of microparticles were as stable to the wash conditions as ones with dense concentrations.

### Example 4

Arrays of clustered arrangements of immobilized microparticles were prepared by creating a slurry of microparticles suspended in a photoreactive polymer and contact printing this slurry on a glass slide. The microparticles were coupled to nucleic acids thereby creating an array of clustered arrangements of microparticles which was used for the detection of the presence of target nucleic acids in a sample.

To fabricate an array of clustered arrangements of oligonucleotide-coupled microparticles, 1 μm streptavidin-coated silica microparticles (CS01N; Bangs Laboratories, Fisher, Ind.) were coupled to biotinylated Cy3-labeled 30-mer oligonucleotides (Integrated DNA Technologies, Coralville, Iowa) by adding 20 μl of a 1.25 μM solution of the oligonucleotide to 100 μl of a 4 mg/ml solution of microparticles in PBS pH 7.5 and allowing the biotin to couple to the streptavidin for 30 minutes at room temperature.

The oligonucleotide-coupled microparticles were then pelleted by centrifugation, the supernatant was removed, fresh deionized water added, and the oligonucleotide-coupled microparticle solution was then vortexed to resuspend the oligonucleotide-coupled microparticles. This wash procedure was repeated twice to remove uncoupled biotinylated oligonucleotide.

The oligonucleotide-coupled microparticles were introduced into a 2.25 mg/mL solution of PV01 (Surmodics, Inc. Eden Prairie, Minn.) in deionized water to form a slurry that contained 4.0 mg/mL microparticles. This slurry was contact printed with a Microgrid II arrayer (Biorobotics, Cambridge, UK) onto a glass slide that had been derivatized with a mixture of n-decyldimethylchlorosilane and tolyldimethylchlorosilane as indicated in Example 1. A pattern of 3×3 spots was repeated on the surface, with the spots having an average diameter of 100 μm and a volume of approximately 0.5-1 pL prior to drying. After printing, the slide was irradiated for two minutes with an ultraviolet light source as detailed in Example 1 (Dymax LightWelder PC2, Dymax Engineering Adhesives, Torrington, Conn.) through a 315 nm cutoff filter (Electro-Lite Corporation, Danbury, Conn.). At this point the microarray was imaged on a fluorescence scanner (Packard Biosciences, ScanArray 5000, Billerica, Minn.) and representative spots were imaged with a fluorescence microscope (Olympus BX 60, Tokyo, Japan). Approximately 100-500 5 μm microparticles were present per 100 μm spot.

Following printing, the array was washed to remove any loosely bound microparticles. The array was washed three times with 1×PBS (pH 7.4) with 0.1% v/v Tween-20, rinsed with deionized water, incubated in a solution of DB02 wash buffer (Surmodics Inc. Eden Prairie, Minn.) for 1 hour at 50° C., followed by two rinses with deionized water. The microarray was then incubated in a solution of 4×SSC/0.1% SDS for two hours at 50° C. and rinsed in deionized water.

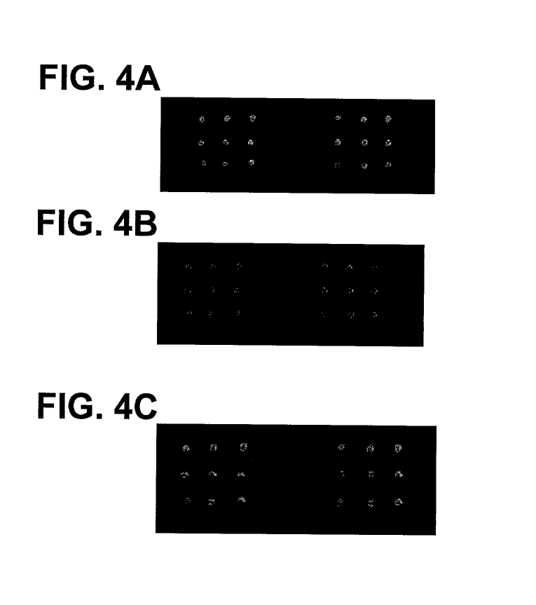

Hybridization of a target nucleotide to the probe oligonucleotide was accomplished by placing a solution containing 500 fmoles of Cy5-labeled complementary oligonucleotide, in 5×SSC and 0.1% SDS, on the array. Hybridization was carried out for 2 hours at 45° C. After hybridization, the microarray was washed with 2×SSC for 5 minutes at 50° C., 0.2×SSC for one minute at room temperature, and finally 0.1×SSC for one minute at room temperature. At this point the microarray was imaged with the fluorescence scanner, using Cy 3 and Cy 5 channels and with the fluorescence microscope. The fluorescence scanner and microscopic images also indicate that the spots on the microarray were clearly visible and contain significant numbers of microparticles, as well the oligonucleotide on the array hybridizes to the complementary oligonucleotide. FIG. 4 shows the clustered arrangement of microparticles on the slide and fluorescence images of the hybridized targets. FIG. 4a shows the microparticle clusters directly after printing; FIG. 4b shows the fluorescence of the clusters of the microparticles in the Cy3 channel (60% laser) after hybridization of the target; FIG. 4c shows fluorescence of the of hybridized Cy5-labeled target oligonucleotide in the Cy5 channel (50% laser).

### Example 5

In this example, arrays were created using the procedure of Example 3 but with decreasing concentrations of photoreactive polymer. At lower concentrations of photoreactive polymer, the polymeric matrix no longer held the microparticles in place following a mild rinse.

Slurries of 5 mg/mL PV01 (SurModics, Inc. Eden Prairie, Minn.) were serially diluted to 5, 2.5, 1.25, 0.625, 0.313 mg/mL in deionized water and 45 μl of each of the serially diluted PV01 solutions were added to 5 μl of 40 mg/ml microparticles in deionized water to create slurries were the final concentration of microparticles is 4 mg/ml in each slurry mixture of 50 μl total.

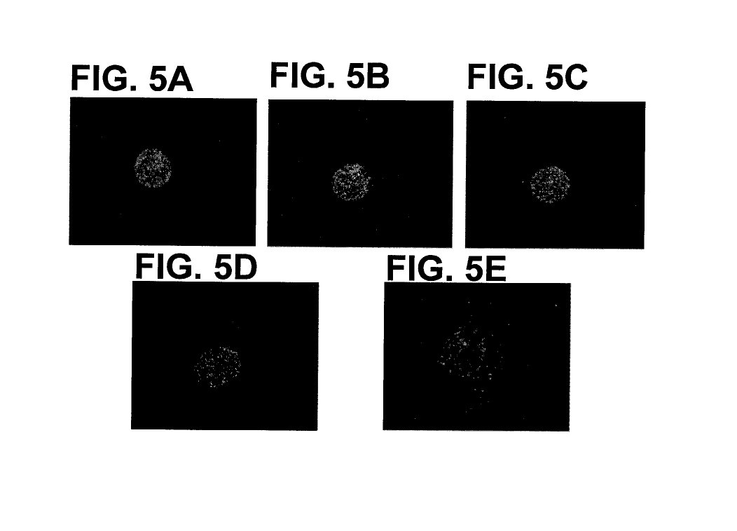

The slurries were all contact printed on a Microgrid II arrayer (Biorobotics, Cambridge, UK) and then irradiated for two minutes with ultraviolet light as detailed in Example 1 (Dymax LightWelder PC2, Dymax Engineering Adhesives, Torrington, Conn.) through a 315 nm cutoff filter (Electro-Lite Corporation, Danbury, Conn.). The arrays were then rinsed three times with 1×PBS with 0.1% Tween-20 and with deionized water. The resulting spots were imaged with a fluorescence microscope (Olympus BX 60, Tokyo, Japan) using 100× magnification. As shown in FIG. 5, the PV01 polymeric matrix immobilized the microparticles until the concentration dropped below 0.625 mg/mL (FIG. 5d), when the microparticles were rinsed free from the polymeric matrix. FIG. 5a shows microparticles immobilized in 5 mg/nL PV01; FIG. 5b shows microparticles immobilized in 2.5 mg/mL PV01, FIG. 5c shows microparticles immobilized in 1.25 mg/mL PV01, FIG. 5d shows microparticles immobilized in 0.625 mg/mL PV01, FIG. 5e shows microparticles immobilized in 0.313 mg/mL mg/mL PV01.

### Example 6

Different matrix-forming materials were used in another method to immobilize microparticles on a substrate. Slurries were prepared, each containing different polymers and microparticles. These slurries were then disposed on a substrate and treated to form matrices in which microparticles were immobilized. Four different photoreactive polymers were used to immobilize 9.9 μm diameter silica microparticles on substrates. These photoreactive polymers PA04, PA05, PV01, and PV05 are all commercially available from SurModics, Inc. (Eden Prairie, Minn.). PA04 and PA05 are copolymers of acrylamide (AA) and N-[3-(4-Benzoylbenzamido)propyl] methacrylamide (BBA-APMA) with differing ratios of BBA-APMA:AA. Similarly PV01 and PV05 are copolymers of vinylpyrrolidone (VP) and N-[3-(4-Benzoylbenzamido)propyl] methacrylamide (BBA-APMA) with differing rations of BBA-APMA:VP. PR03 (as described in Example 4) a non-polymeric photoreactive material was also used in this Example.

PA04, PA05, PV01, PV05, and PR03 were dissolved in deionized water at a concentration of 2.5 mg/ml. 100 μL of each solution containing photoreactive compound was added to a pellet of 2 mg of 9.9 μm diameter silica microparticles (SS06N, Bangs Laboratories, Fisher, Ind.) that had been washed three times with deionized water to create a slurry. The final concentration of each slurry was 2.5 mg/ml of photoreactive compound and 20 mg/ml of microparticles. Each slurry was printed with 25 gauge disposable needles (PrecisionGlide Needles, Becton Dickinson and Co., Franklin Lakes, N.J.) and an x-y programmable stage (CAMM-3, Roland Digital Group, Irvine, Calif.) onto glass microscope slides which had been functionalized with silanes, as detailed in Example 1 and onto acrylic slides (Cadillac Plastics, Minneapolis, Minn.). This printing forms a pattern of approximately 300-400 μm diameter spots on the substrates.

The coated substrates were irradiated for two minutes with ultraviolet light as detailed in Example 1. The coated substrates were then imaged with a fluorescence microscope (Olympus BX 60, Tokyo, Japan), to ensure that patterning was successful.

Following this, the coated substrate was washed to remove any loosely bound microparticles. The coated substrates were washed three times with 1×PBS (pH 7.4) with 0.1% v/v Tween-20, rinsed with deionized water. At this point, each was again imaged with the fluorescence microscope to determine microparticle loss in the various photoreactive polymer matrices. After imaging, the coated substrates were incubated in a solution of DB02 wash buffer (Surmodics Inc. Eden Prairie, Minn.) for 1 hour at 50° C., followed by two rinses with deionized water. The coated substrates were then incubated in a solution of 4×SSC/0.1% SDS for two hours at 50° C. and rinsed in deionized water. Presence of the microparticles before and after washing steps are summarized in Table 4.

TABLE 4

| | | Presence | | |
|---|---|---|---|---|
| | | of micro- | Presence of | Presence of |
| | | particles | microparticles | microparticles |
| Photoreactive | | before | after PBS- | after high salt |
| Compound | Substrate | washes | Tween wash | wash (4X SSC) |
| PA04 | Glass | Present | No loss | No loss |
| PA04 | Acrylic | Present | No loss | No loss |
| PA05 | Glass | Present | No loss | No loss |
| PA05 | Acrylic | Present | No loss | Some loss* |
| PV01 | Glass | Present | No loss | No loss |
| PV01 | Acrylic | Present | No loss | No loss |
| PV05 | Glass | Present | No loss | No loss |
| PV05 | Acrylic | Present | No loss | No loss |
| PR03 - non- | Glass | Present | No loss | Complete loss |
| polymer | | | | |
| control | | | | |
| PR03 - non- | Acrylic | Present | No loss | Complete loss |
| polymer | | | | |
| control | | | | |

*Sample was touching another slide during the wash.

We claim:

1. A method for making an array, the method comprising steps of:
a) preparing at least one slurry comprising
i) a matrix-forming material, and
ii) a plurality of microparticles wherein the microparticles of each plurality comprise a probe coupled to the microparticle, wherein the probe is configured and arranged to specifically bind a target;
b) disposing the at least one slurry on a substrate to form at least one clustered arrangement of microparticles; and
c) treating the slurry so the matrix-forming material forms a matrix, wherein the microparticles become immobilized in the matrix of the clustered arrangement on the substrate.

2. The method of claim 1 wherein the matrix-forming material comprises a polymer.

3. The method of claim 2 wherein the polymer comprises a reactive polymer.

4. The method of claim 3 wherein the reactive polymer comprises a photoreactive polymer having at least one photoreactive group and the photoreactive group is selected from the group consisting of aryl ketones, arylazides, acyl azides, sulfonyl azides, phosphoryl azides, diazoalkanes, diazoketones, diazoacetates, and ketenes.

5. The method of claim 3 wherein the reactive polymer comprises polymers selected from the group consisting of functionalized polyacrylamide, polymethacrylamide, polyvinylpyrrolidone, polyacrylic acid, polyethylene glycol, polyvinyl alcohol, poly(HEMA), copolymers thereof, and combinations thereof.

6. The method of claim 3 wherein the reactive polymer i s selected from the group of photoreactive copolymers consisting of vinylpyrrolidone and N-[3-(4-Benzoylbenzamido)propyl] methacrylamide (BBA-APMA); and acrylamide and BBA-APMA.

7. The method of claim 3 wherein the reactive polymer comprises polymers selected from the group consisting of functionalized polysaccharides, glycosaminoglycans, polypeptides, and combinations thereof.

8. The method of claim 1 wherein the step of treating, the microparticles become immobilized by entrapment in the matrix, wherein the entrapment of the microparticles does not depend on the formation of ionic or covalent bonds between the microparticle and the matrix forming material.

9. The method of claim 1 wherein the step of treating comprises irradiating the slurry with electromagnetic energy.

10. The method of claim 1 wherein each clustered arrangement of microparticles comprises a unique probe.

11. The method of claim 1 wherein the step of disposing comprises spotting at least one slurry on the substrate.

12. The method of claim 1 wherein the probe comprises a nucleic acid.

13. The method of claim 1 wherein the probe comprises a polypeptide.

14. The method of claim 13 wherein the polypeptide comprises an antibody or fragment thereof.

15. The method of claim 1 wherein the step of disposing comprises applying the slurry to the substrate by pin printing or jet printing.

16. An array comprising:
a) a substrate; and
b) at least one clustered arrangement of microparticles comprising a plurality of microparticles immobilized in a matrix on the substrate, wherein the microparticles of each plurality comprise a probe coupled to the microparticle, wherein the probe is configured and arranged to specifically bind a target.

17. The array of claim 16 wherein the matrix-forming material comprises a polymer.

18. The array of claim 17 wherein the polymer comprises a reactive polymer.

19. The array of claim 18 wherein the reactive polymer comprises a photoreactive polymer having at least one photoreactive group and the photoreactive group is selected from the group consisting of aryl ketones, arylazides, acyl azides, sulfonyl azides, phosphoryl azides, diazoalkanes, diazoketones, diazoacetates, and ketenes.

20. The array of claim 18 wherein the reactive polymer comprises polymers selected from the group consisting of functionalized polyacrylamide, polymethacrylamide, polyvinylpyrrolidone, polyacrylic acid, polyethylene glycol, polyvinyl alcohol, poly(HEMA), copolymers thereof, and combinations thereof.

21. The array of claim 18 wherein the reactive polymer is selected from the group of photoreactive copolymers consisting of vinylpyrrolidone and N-[3-(4-Benzoylbenzamido)propyl] methacrylamide (BBA-APMA); and acrylamide and BBA-APMA.

22. The array of claim 18 wherein the reactive polymer comprises polymers selected from the group consisting of functionalized polysaccharides, glycosaminoglycans, polypeptides, and combinations thereof.

23. The array of claim 16 wherein the immobilization of the microparticles comprises entrapment of the microparticles in the matrix, and the entrapment of the microparticles does not depend on the formation of ionic or covalent bonds between the microparticles and the matrix forming material.

24. The array of claim 16 wherein each clustered arrangement of microparticles comprises a unique probe.

25. The array of claim 16 wherein the probe comprises a nucleic acid.

26. The array of claim 16 wherein the probe comprises a polypeptide.

27. The array of claim 26 wherein the polypeptide comprises an antibody or fragment thereof.

28. A method for detecting a target in a sample, the method comprising steps of:
a) providing an array comprising
i) a substrate; and
ii) at least one clustered arrangement of microparticles comprising a plurality of microparticles immobilized in a matrix on the substrate, wherein the microparticles of each plurality comprise a probe coupled to the microparticle, wherein the probe is configured and arranged to specifically bind a target;
b) applying a sample suspected of containing the target to the array;
c) maintaining the sample and array under conditions to allow binding of the target to the probe; and
d) detecting 1) the a target marker coupled to the target and associated with the clustered arrangement, and 2) the location of the clustered arrangement, thereby determining the presence or amount of the target in the sample.

29. The method of claim 28 wherein the matrix-forming material comprises a polymer.

30. The method of claim 29 wherein the polymer comprises a reactive polymer.

31. The method of claim 30 wherein the reactive polymer comprises a photoreactive polymer having at least one photoreactive group and the photoreactive group is selected from the group consisting of aryl ketones, arylazides, acyl azides, sulfonyl azides, phosphoryl azides, diazoalkanes, diazoketones, diazoacetates, and ketenes.

32. The method of claim 30 wherein the reactive polymer comprises polymers selected from the group consisting of functionalized polyacrylamide, polymethacrylamide, polyvinylpyrrolidone, polyacrylic acid, polyethylene glycol, polyvinyl alcohol, poly(HEMA), copolymers thereof, and combinations thereof.

33. The method of claim 30 wherein the reactive polymer is selected from the group of photoreactive copolymers consisting of vinylpyrrolidone and N-[3-(4-Benzoylbenzamido)propyl] methacrylamide (BBA-APMA); and acrylamide and BBA-APMA.

34. The method of claim 30 wherein the reactive polymer comprises polymers selected from the group consisting of functionalized polysaccharides, glycosaminoglycans, polypeptides, and combinations thereof.

35. The method of claim 28 wherein immobilization comprises entrapment in a matrix, and entrapment of the microparticles does not depend on the formation of ionic or covalent bonds between the microparticle and the matrix forming material.

36. The method of claim 28 wherein each clustered arrangement of microparticles comprises a unique probe.

37. The method of claim 28 wherein the probe comprises a nucleic acid.

38. The method of claim 28 wherein the probe comprises a polypeptide.

39. The method of claim 38 wherein the polypeptide comprises an antibody or fragment thereof.
